(19) United States
(12) Patent Application Publication
(10) Pub. No.: US 2004/0171594 A1
(43) Pub. Date: Sep. 2, 2004

(54) Use of bis-acid(5-androsten-17-one-3beta-hydroxyl)diester for manufacture pharmaceuticals

(76) Inventors: Donghui Xu, Guangzhou (CN); Shibo Xu, Guangzhou Guangdong (CN)

Correspondence Address: EPSTEIN DRANGEL BAZERMAN & JAMES, LLP, 60 EAST 42ND STREET, SUITE 820, NEW YORK, NY, 10165, US

(21) Appl. No.: 10/480,173

(22) PCT Filed: May 29, 2002
(86) PCT No.: PCT/CN02/00364

(30) Foreign Application Priority Data
Jun. 28, 2001 (CN) 0114864.0

(51) Int. Cl. A61K 31/56

(52) U.S. Cl. 514/169

(57) ABSTRACT

The invention relates to the application of diacid-(5-androsten-17-one-3β-ol)-diester (formula I) as drug manufacture for arrhythmia, myocardial ischemia, cerebral ischemia, osteoporosis, renal failure and so on.

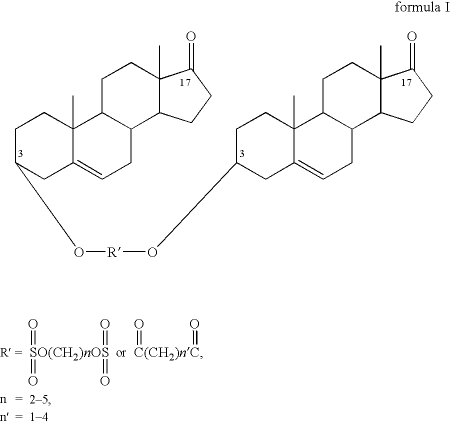

### TECHNIQUE DOMAIN

The invention related to application of diacid-(5-androsten-17-one-3 β-ol)-diester (formula I) in pharmacy fields.

### RESEARCH BACKGROUND

With improvement of the living level and medical health measures, the average human lifetime X, becomes longer and longer. However, problems of the senile become more remarkable than anytime before. According to the research in Senile Institute of China, the population of the aged has rapidly increased at an average rate of 3.32% per year since 1990. Until the end of 1994, the amount of people aged over sixty in China had reached 110 million, which was equivalent to 9.5% of the total population. China has become an “senile” country due to the continuous increase of senile population after 2001. Nowadays the problem of prevetion and cure of senile diseases becomes more and more serious. Therefore to develop new drugs for senile diseases is one of hot spots in medical fields.

Arrhythmia, myocardial ischemia, cerebral ischemia, hypoimmunity, osteoporosis, renal failure and so on are popular senile diseases and sometimes it shows syndrome for senile poeple. Limited by single effect on only one certain disease for most medicines, lots of cases haven't achieved satisfactory curative results yet. Thus developing a new kind of new drug with favorable complex effects on multi-agedness diseases mentioned above will be very important to the health of senile people.

### INVENTION CONTENT

This invention aim is to apply diacid-(5-androsten-17-one-3β-ol)-diester as drugs to cure popular senile diseases such as arrhythmia, myocardial ischemia, cerebral ischemic, hypoimmunity, osteoporosis and renal failure.

The structure of diacid-(5-androsten-17-one-3β-ol)-diester is described as formula I (abbrev. compounds I)

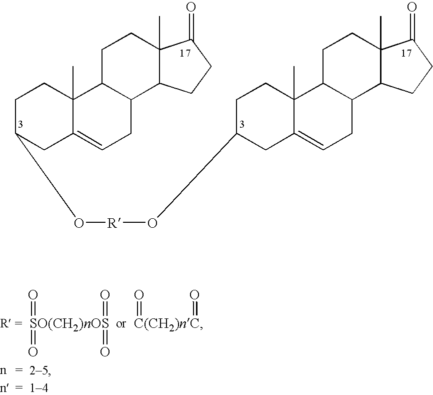

According to the different R′ groups, compounds I can be divided into two types: one is dialcohol-disulphonic acid-(5-androsten-17-one-3β-ol)-diester (abbrev. Compounds I A), such as glycol-d isulphonic acid-(5-androsten-17-one-3β-ol)-diester, propanediol-disulphonic acid-(5-androsten-17-one-3β-ol)-diester, butanediol-disulphonic acid-(5-androsten-17-one-3β-ol)-diester, pentanediol-disulphonic acid-(5-androsten-17-one-3β-ol)-diester; The other is dicarboxylic-(5-androsten-17-one-3β-ol)-diester (abbrev. compounds I B), such as malonic acid-(5-androsten-17-one-3β-ol)-diester, succinic acid-(5-androsten-17-one-3β-ol)-diester, glutaric acid-(5-androsten-17-one-3β-ol)-diester or adipic acid-(5-androsten-17-one-3β-ol)-diester.

The applications of the inventation include follow fields:

The application of the compounds I on the drug manufacture for curing arrhythmia;

The application of the compounds I on curing myocardial ischemia, cerebral ischemia;

The application of the compounds I compounds on enhancing the immunity;

The application of the compounds I compounds on curing osteoporosis and renal failure.

The most effective compound in compounds I is succinic acid-(5-androsten-17-one-3β-ol)-diester (formulla II).

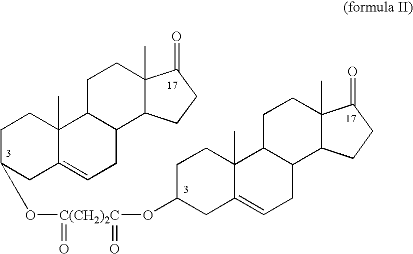

It has been experimentally proved that compounds I have remarkable effects on curing arrhythlimiia, myocardial ischemia, cerebral ischemia, hypoimmunity, osteoporosis, renal failure and so on. Therefore, we expect the drugs with compounds I have favorable therapeutic effects on those relative diseases. Our previous research proved the potential for compounds I applied as drugs.

Besides their excellent therapeutic effects, the compounds I have also shown the low toxicity and high security as drugs.

To be manufactured as drugs, compounds I can be assorted with the ordinary filling material through routine methods. According to the requirements, they can be made in different forms, such as injection, transfusion, tablet, powder, granule, capsule, sirup or suppository agent. The medicines with compounds I as bioactivity are usually taken by oral administration and can adopt taking methods as well. The dose range varies from 0.01 to 1000 mg a day. The common dose for adults is between 0.1 mg and 500 mg per day. The maximum dose is 0.3 to 300 mg a day, being used once or manifold a day.

The compounds IA of the invention can be made by dissolving sodium 5-androsten-17-one-3-sulphonate (formulaIII) into the glycol dimethyl ether, followed by dihaloalkyl [di-iodo(or bromo)ethane (or propane, butane, pentane)](formula IV) and reacting with the phase transfer agent (n-butyl bromo-amine). The whole reaction routine was shown as below:

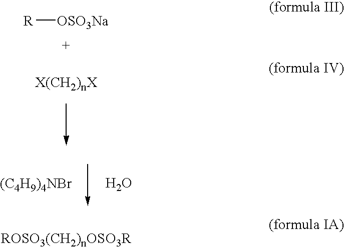

X=I or Br, n=2˜5, R=C₁₉H₂₇O, n in formula IV is the same as n in formula I

The compounds IB can be prepared by the reaction of 5-androsten-17-one-3β-ol (formulaV) with appropriate diacylchlorine (as propane, butane, pentane or hexane dioyl dichloride) (formula VI) under stirring in tetrahydrofuran, 110° C. The procedure is described as below:

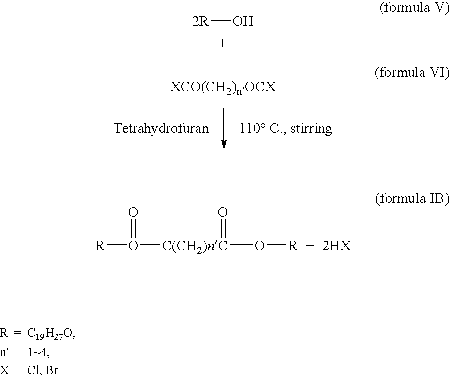

Concretely Actualizing Examples of the Invention

Preparation details of compounds I compounds are stated with following examples:

### EXAMPLE 1

Weighed 3.68 g sodium-(5-androsten-17-one-3β-ol)-sulphonate and dissolved it with 50 mL glycoldimethyl ether. Then added 1.5 g 1,2-diiodo-ethane, 1.6 g n-tetrabutyl bromo-amine and 20 mL water. The reaction was under stirring and reflux for 4 hr at 60° C. After cooling, extracted the mixture with ether for three times. The extracted phase was washed with water and then dried with anhydrous sodium sulfate. Evaporated solvent and obtained expected glycol-disulphonic acid-(5-androsten-17-one-3β-ol)-diester (6.1 g).

The final products could be propanediol-disulphonic acid-(5-androsten-17-one-3β-ol)-diester, butanediol-disulphonic acid-(5-androsten-17-one-3β-ol)-diester, pentanediol-disulphonic acid-(5-androsten-17-one-3β-ol)-diester if 1,2-diiodo-ethane was respectively alternated by 1,3-diiodo-propane 1,4-diiodo-butane or 1,5-diiodo-pentane.

### EXAMPLE 2

Dissolved 500 g 3β-ol-5-androsten-17-one with 1000 mL tetrahydrofuran and then dropping 50 mL butanedioyl dichloride. Refluxed the mixture with stirring at 110° C., by TCL method to determine the reaction end. The crude product was obtained by pouring the mixture into ice water and washing with ethyl acetate/petreolum (1:1) for three times. The crude product was chromatographed on a Si gel H column to give pure products of formulacompounds II (400 g).

The final products could be malonic acid-(5-androsten-17-one-3β-ol)-diester, adipic acid-(5-androsten-17-one-3β-ol)-diester or glutaric acid-(5-androsten-17-one-3β-ol)-diester, according to the butanedioyl diochloride alternation by propanedioyl dichloride, hexanedioyl dichloride or pentanedioyl dichloride, respectively.

In the following part, the medical purpose and effect of compounds I will be further demonstrated with experiment examples.

### EXPERIMENT EXAMPLE 1

### Acute Toxin Experiment

1. Materials

Experiment specimens: The compounds I (totally eight species: compounds IA, n=2,3,4,5 and compounds I B n′=1,2,3,4) were supplied by the Research Center of Physiological Activity of Natural Products, Shizhen Hall, Guangzhou. Certificate No. were 19970101, 19970102, 19970103, 19970104, 19970105, 19970106, 19970107, 19970108, respectively.

Experiment animals: NIH breed rats were supplied by the Animal Center of Medical Experiment of Guanlgdong Province. Certificate No. 1997A029.

2. Methods and Results

The above eight compounds I were tested, respectively:

2.1 Acute Toxicity Experiment by Intragastric Intubation of Specimens

Chose twenty NIH mice, weighing 20±2 g, equal amount of male and female. The specimen was mixed in 0.5% CMC-Na solution to be suspending solution, administered orally mice with 0.8 mL/20 g body weight once a day. Under continuous observation for seven days, mice moved agilely with their fur being glazed. Neither death nor abnormal reaction was aroused. Limited to concentration and volume of drug administration could not be increased more, value of LD₅₀ could not be tested. The maximum tolerance dose experiment was performed. Chose other 20 healthy mice, equal amount of male and female. Mice were administered orally suspending solution of the compounds I (20 mg/mL) for three times within 24 hours, with 0.5 mg/20 g body weight and the total dose was 1.5 g/kg body weight. The observation continued for seven days and let mice drink and eat freely. They moved agilely with their fur being glazed. Neither death nor abnormal reaction was aroused.

2.2 Acute Toxicity Experiment by Abdominal Injection of Specimens

Chose twenty healthy NIH breed mice, equal amount of male and female. The specimen was mixed in 0.5% CMC-Na solution to be suspending solution, treated into tiny and even grains with Ultrasonic Emulsion and High Speed Agitator. The mice were injected abdominally specimen with 1.0 mg for per 20 g body weight once a time. During continuous observation for seven days, mice moved agilely with their fur being glazed. Neither death nor abnormal reaction was aroused. Limited to concentration and volume of drug administration could not be increased more, value of LD₅₀ could not be tested. The maximum tolerance dose experiment was performed. Chose other 40 healthy NIH breed mice, equal amount of male and female. The specimen was administered abdominally with 20 mg/mL every 4 hours within 24 hours, 0.5 mL per 20 g body weight once a time. During observation continued for seven days, the mice drunk and ate freely.

3. Conclusions

3.1 The maximum tolerant dose of mice administered orally the formula I compounds per day were no less than 1.5 g/kg (=0.5×3×20×50). According to the conversion of area of body surface, the maximum tolerant dose were equal to 232.7 times (=0.5×3×20×387.9÷50) of 70 kg adult with dose 50 mg. During the observation period of the maximum tolerant dose experiment, mice moved agilely with their fur being glazed. Neither death nor abnormal reaction was aroused.

3.2 The maximum tolerant dose of mice injected abdominally with the compounds I was no less than 3 g/kg body weight (=0.5×20×6×50 mg). During observation period of maximum tolerant dose experiment, mice moved agilely with their fur being glazed. Neither death nor abnormal reaction was aroused.

The maximum tolerant dose of mice injected bdominally with compounds I were equal to 232.7 times (0.5×6×20×387.9÷100) of a 70 kg adult's dose with 10 mg a day.

3.3 Based on above analyses, the compounds I can be considered as notoxic compounds.

### EXPERIMENT EXAMPLE 2

### Antiarrhythmic Function

1. Experiment Materials

malonic acid-(5-androsten-17-one-3β-ol)-diester (code-name compound C), succinic acid-(5-androsten-17-one-3β-ol)-diester (code-name compound D), glutaric acid-(5-androsten-17-one-3β-ol)-diester (code-name compound E), adipic acid-(5-androsten-17-one-3β-ol)-diester (code-name compound F), glycol-disulphonic acid-(5-androsten-17-one-3β-ol)-diester (code-name compound G), propanediol-disulphonic acid-(5-androsten-17-one-3β-ol)-diester (code-name compound H), butanediol-disulphonic acid-(5-androsten-17-one-3β-ol)-diester (code-name compound I) and pentanediol-disulphonic acid-(5-androsten-17-one-3β-ol)-diester (code-name compound J) were synthesized in our laboratory. These were mixed with 0.5% CMC-Na to be different concentration when they were used; Prasterone (Pras) and Amiodarone (Amio) were manufactured by Sanofi & Winthrop drug factory; Acontine (Aco) and Ouabain (Oua) were produced by Merck Co.; CMC-Na was imported and repacked by Wholesale Department of Chemical Agent and Glass Apparatus, Guangzhou.

NIH breed mice were supplied by the Animal Center of Medical Experiment, Guangdong Province. Certificate No: 97A018.

SD rats were supplied by the Animal Center of Medical Experiment, Guangdong Province. Certificate No. 97A017.

New Zealand white rabbits were supplied by Weilong breed factory of Huangqi Michong, Nanhai of Guangdong Province, Certificate No:26-97043.

MS-302 Multimedia Biological Signal Record and Analysis System (Producted by Pharmacy College of Guangdong, China); Microinfusion Pump WZ-50D (Producted by Medical Instruments of Zhej iang Medicine University).

2. Methods and Results

2.1 Effect on Arrhythmia of Rats Induced by Aconitine

Male SD rats, weighing (218.3±20.0) g, were anaesthetized with urethane 1.2 g/kg body weight by abdominal injection, continuously monitored by II lead electrocardiogram (ECG) by MS302 Biological Signal Record and Analysis System. After ten minutes of being administered drug or 0.5% CMC-Na through sublingual vein, rats were injected with Aconitine in external jugular vein at 2.5 μg/min speed, recorded the dose at appearance time of ventricular premature beat (VP), ventricular tachycardia (VT), ventricular fibrillation (VF) and cardiac arrest (CA), respectively. And t test was performed. The results showed as in Table 1 that Aconitine dose leading to appearance of VP, VT, VF and CA in groups administered compounds C, D and E of formula I compounds were higher than that of gounp administered 0.5% CMC-Na, respectively. The effect of compounds Ca Do E at 0.5 mg/kg dose were remarkably better than that of Prasterone and Arniodarone.

TABLE 1
Effect on arrhythmia of rats induced by Aconitine (n = 10, x ± s)

| Group | Dose of Aconitine(μg · kg⁻¹) required to induce | | | |
|---|---|---|---|---|
| Dose/mg · kg⁻¹ | VP | VT | VF | CA |
| CMC-Na | 65.5 ± 15.2 | 84.6 ± 30.1 | 121.4 ± 28.0 | 148.2 ± 33.0 |
| Compound C 0.5 | 98.0 ± 29.1** | 118.9 ± 35.3* | 152.8 ± 39.3* | 186.5 ± 31.7* |
| Compound D 0.5 | 122.4 ± 20.5** | 137.8 ± 24.4* | 186.4 ± 30.2* | 204.0 ± 28.0* |
| Compound E 0.5 | 108.2 ± 37.4** | 122.4 ± 37.8* | 163.4 ± 30.5** | 184.5 ± 27.1* |
| Parsterone 4.5 | 93.5 ± 19.0** | 110.0 ± 16.1* | 132.7 ± 24.4 | 164.5 ± 27.1 |
| Amiodarone 3 | 90.0 ± 25.7* | 108.8 ± 25.4 | 141.2 ± 28.2 | 176.0 ± 38.0* |

*p < 0.05,
**p < 0.01 vs CMC-Na group

2.2 Effect on Arrhythmia of Guinea Pigs Induced by Ouabain

Chose 40 male guinea pigs, weighing (288.8±38.3) g, randomly divided into six groups, anaesthetized with urethane 1.2 g/kg by abdominal injection. Ten minutes after administered drug or 0.5% CMC-Na, the guinea pigs were injected Ouabain into external jugular vein at 3 μg/min speed, recorded ECG respectively, measured the dose at appearance time of VP, VT, VF and CA, and performed t test. Table 2 showed that the needed Ouabain doses of appeared VP, VT, VF and CA were remarkably increased when administered compounds F, G and H respectively, which indicating that compounds F, G and H could enhance the tolerant dose of Oua which induced toxic heart of guinea pigs.

TABLE 2
Effect on arrhythmia of guinea pigs induced by Oua (n = 8, x ± s)

| | Dose of Oua | | | |
|---|---|---|---|---|
| Group Dose/ | (μg/kg) | | | |
| mg · kg⁻¹ | VP | VT | VF | CA |
| CMC-Na | 60.9 ± 14.6 | 92.9 ± 29.0 | 137.2 ± 31.9 | 166.6 ± 33.7 |
| Compound F 1.5 | 87.4 ± 21.3** | 139.4 ± 35.4** | 176.2 ± 48.1* | 212.0 ± 40.6* |
| Compound G 1.5 | 110.9 ± 20.3*** | 144.3 ± 33.8** | 188.7 ± 37.8** | 228.0 ± 39.8** |
| Compound H 2.5 | 116.0 ± 37.0** | 140.2 ± 35.6** | 198.1 ± 41.5** | 223.2 ± 30.7** |
| Parsterone 4.5 | 89.3 ± 15.5*** | 131.6 ± 24.6** | 165.4 ± 26.4* | 195.5 ± 33.0* |
| Amiodarone 3 | 118.4 ± 33.5*** | 175.0 ± 41.1*** | 247.6 ± 46.1*** | 283.6 ± 43.9* |

*p < 0.05,
**p < 0.01,
***p < 0.001 vs CMC-Na group

2.3 Effect on Arrhythmia of Rat Induced by BaCl₂

50 male SD rats, weighing (236.7±13.7)g, were anaesthetized with 10% chloral hadrate 0.3 g/kg intraperitoneally, recorded ECG with intubating of external jugular vein administering drug. As soon as injected with BaCl₂ 4 mg/kg, rats usually emerged VP and VT quickly and developed to be the steady and typical double-aspect arrhythmia. After immediately injected with drug or 0.5% CMC-Na into vein, recorded the time of recovering and maintaining sinus rhythm within 60 min as standard, and compared the function between five groups. Show as Table 3, compound I and J showed having remarkably therapeutic function to arrhythmia induced by BaCl₂. Time of maintaining sinus rhythm was longer and acted more quickly than that of Amiodarone group.

TABLE 3
Effect on arrhythmia of rats induced by BaCl₂ (n = 10, x ± s)

| | time of storing | time of maintaining |
|---|---|---|
| Groups | sinus rhythm | sinus rhythm |
| (mg/kg) | (min) | (min) |
| CMC-Na | 38.648 ± 8.142 | 21.482 ± 8.041 |
| Compound I 0.5 | 6.752 ± 4.132*** | 45.478 ± 5.271*** |
| Compound J 1.5 | 3.584 ± 2.471*** | 51.762 ± 6.275*** |
| Compound J 2.5 | 0.845 ± 1.529*** | 59.248 ± 1.527*** |
| Amiodarone 3 | 15.859 ± 5.126*** | 40.156 ± 5.124*** |

***p < 0.001, vs CMC-Na group

### EXPERIMENT EXAMPLE 3

### Protective Function to Myocardial Ischemia

1. Experiment Materials

malonic acid-(5-androsten-17-one-3β-ol)-diester (code-name compound C), succinic acid (5-androsten-17-one-3β-ol)-diester (code-name compound D), glutaric acid-(5-androsten-17-one-3β-ol)-diester (code-name compound E), adipic acid-(5-androsten-17-one-3β-ol)-diester (code-name compound F), glycol-disulphonic acid-(5-androsten-17-one-3β-ol)-diester (code-name compound G), propanediol-disulphonic acid-(5-androsten-17-one-3β-ol)-diester (code-name compound H), butanediol-disulphonic acid-(5-androsten-17-one-3β-ol)-diester (code-name compound 1) and pentanediol-disulphonic acid-(5-androsten-17-one-3β-ol)-diester (code-name compound J) were synthesized by Medicinal Chemistry Lab, Pharmacy Department of Zhongshan University. These were mixed with 0.5% CMC-Na to be different concentration when they were used; Nifedipine was manufactured by Liqun Pharmacy Co. of Datong City; CMC-Na was imported and repacked by Wholesale Department of Chemical Agent and Class Apparatus, Guangzhou.

SD rats were supplied by the Animal Center of Medical Experiment of Guangdong Province. Certificate NO. 98A017.

New Zealand rabbits were supplied by Weilong Freed Farm of Huangqi Michong, Nanhai of Guangdong Province, No.26-98043.

MS-302 Multimedia Biological Signal Record and Analysis System (Guangdong Pharmacy College); Microinfusion Pump WZ-50D (Medical Instruments Factory of Zhejiang Medicine University).

2. Experiment Methods

SD rats were measured ECG by chest leads before experiment, abandoned those of abnormal or arrhythmia at T wave, ST sect. Choosing qualified rats, weighing (208.1±18.8)g, both male and female were used in the experiment. They were divided into four groups according to Table 4, eight rats in each group. As dose of Table 4, tested compounds or 0.5% CMC-Na solution with same volume were administered through duodenum for 30 minutes, rats were anaesthetized with aether. After ECG was recorded, Isoproterenol (ISO) was administered by subcutaneous injection from the back, recorded ECG again after 30 minutes. After specimen was intragastricly granted for 24 hours and 48 hours, every group was administered simples again and recorded ECG again in terms of the methods stated above. After 72 hours, bleeding from the ophthalmic venous plexus and separated blood serum through centrifugation. Killed the rats, scissor the tip of the heart to prepare 10% myocardial tissue homogenate with 4° C. physiological saline as standby.

Effect on Displacement Extent of ΣST Interval

Measured each rat's value of ST before first administering ISO. And after first, second and third subcutaneous injection for 30 minutes, calculated mv mean value of ΣST as guide line of myocardium injury and performed t test.

Effect on Creatine Phokinase (CK), Lactate Dehydrogenase (LDH) and Malondialdegyde (MDA) in Serum

The changes of MDA LDH and CK in serum were measured with agent kits.

Effect on CK, LDH and MDA in Myocardial Tissue Homogenate

The changes of MDA, LDH and CK in serum and myocardial tissue homogenate were measured with agent kits.

3. Results

3.1 Effect on ΣST Interval

The change extent of ST interval could basically reflect severe extent of myocardial ischemia. The ΣST interval could act as the ration guide line of the ischemia extent. After inducing myocardial ischemia of rats by subcutaneous injection with ISO, ST interval remarkably enhanced and showed that myocardium of rats had severe injury of myocardial ischemia. The results of experiment (see Table 4) indicated that our synthetic compounds and Nifedipine could decrease abnoenal enhance extent (average displacement range of ΣST) of ST interval induced by subcutaneous injection with ISO. The compounds I had protective function to rats myocardium from myocardial ischemia induced by ISO.

TABLE 4
Effect on Σ ST in myocardium injury model of rats induced by ISO

| Group Dose/ | Σ ST/mv | | | |
|---|---|---|---|---|
| mg · kg⁻¹ | 30 min | 24 h | 48 h | 72 h |
| CMC-Na 0.76 | 1.26 ± 0.24 | 1.18 ± 0.21 | 0.87 ± 0.17 | 0.90 ± 0.15 |
| Compound C 0.76 | 0.64 ± 0.21*** | 0.52 ± 0.11*** | 0.50 ± 0.14*** | 0.52 ± 0.12*** |
| Compound D 0.76 | 0.42 ± 0.20*** | 0.47 ± 0.13*** | 0.42 ± 0.08*** | 0.40 ± 0.10*** |
| Compound E 0.76 | 0.53 ± 0.11*** | 0.50 ± 0.10*** | 0.57 ± 0.04*** | 0.51 ± 0.07*** |
| Compound F 0.76 | 0.47 ± 0.10*** | 0.57 ± 0.15*** | 0.61 ± 0.05*** | 0.53 ± 0.08*** |
| Compound G 0.76 | 0.51 ± 0.13*** | 0.50 ± 0.14*** | 0.67 ± 0.19*** | 0.42 ± 0.07*** |
| Compound H 0.76 | 0.53 ± 0.11*** | 0.50 ± 0.10*** | 0.52 ± 0.04*** | 0.51 ± 0.12*** |
| Compound I 0.76 | 0.49 ± 0.12*** | 0.54 ± 0.12*** | 0.50 ± 0.06*** | 0.54 ± 0.08*** |
| Compound J 0.76 | 0.54 ± 0.12*** | 0.52 ± 0.07*** | 0.56 ± 0.07*** | 0.54 ± 0.08*** |
| Nif 0.76 | 0.50 ± 0.09*** | 0.53 ± 0.08*** | 0.52 ± 0.05*** | 0.51 ± 0.07*** |

\overline{x} ± s, n = 8,
***p < 0.001, vs CMC-Na group

3.2 Effect on CK LDH and MDA in Serum

After subcutaneous injection with ISO, indexes of CK, LDH and MDA in rat serum remarkably increseed and showed that CK and LDH of myocardial cells omitted into blood due to injury of inyocardial tissue, and Lipid Per-Oxidation (LPO) in serum increased largely. Table 5 showed that specimen could remarkably inhibit CK and LDH omitting from myocardial cell to serum, as well as could remarkably inhibit MDA of rat serum abnormally enhancing induced by ISO and had protective function to myocardial cells.

TABLE 5
Effect on serum of rats with myocardium injury

| | Activity of CK | | MDA |
|---|---|---|---|
| Group Dose/mg · kg⁻¹ | (U/L) | Activity of LDH (U/L) | (nmol/mL) |
| CMC-Na 0.76 | 359.23 ± 34.25 | 389.23 ± 34.41 | 50.42 ± 3.21 |
| Compound C 0.76 | 262.50 ± 35.41*** | 270.13 ± 27.46*** | 28.29 ± 6.56*** |
| Compound D 0.76 | 229.27 ± 31.32*** | 218.55 ± 37.35*** | 18.15 ± 4.52*** |
| Compound E 0.76 | 275.44 ± 21.81*** | 267.35 ± 38.42*** | 25.54 ± 3.80*** |
| Compound F 0.76 | 252.17 ± 18.41*** | 250.40 ± 31.30*** | 24.16 ± 4.18*** |
| Compound G 0.76 | 248.20 ± 20.50*** | 254.75 ± 30.81*** | 20.54 ± 2.11*** |
| Compound H 0.76 | 261.24 ± 24.27*** | 253.24 ± 31.55*** | 21.17 ± 3.36*** |
| Compound I 0.76 | 242.14 ± 22.72*** | 233.35 ± 18.46*** | 24.64 ± 2.53*** |
| Compound J 0.76 | 239.40 ± 16.15*** | 257.05 ± 31.30*** | 26.47 ± 3.25*** |
| Nif 0.76 | 255.90 ± 31.22*** | 224.01 ± 51.20*** | 27.17 ± 3.44*** |

x ± s, n = 8,
***p < 0.001, vs CMC-Na,

3.3 Effect on CK, LDH and MDA in Myocardial Tissue Homogenate

After subcutaneous injection with ISO, the content of CK and LDH in myocardial tissue remarkably decreased, but the content of MDA in myocardial tissue remarkably enhanced. The results of experiment in Table 6 showed that compared with CMC-Na group, Nifedipine group and groups of the formula I compounds could remarkably inhibit CK and LDH omitting from cells and the compounds I had remarkably protective function to injury of myocardial tissue.

TABLE 6
effect on myocardial tissue homogenate of rats
with myocardium injury (x ± s, n = 8)

| | Activity of CK | Activity of LDH | MDA |
|---|---|---|---|
| Group Dose/mg · kg⁻¹ | (U/L) | (U/L) | (nmol/g) |
| CMC-Na 0.76 | 108.84 ± 21.11 | 98.57 ± 12.81 | 253.23 ± 35.11 |
| Compound C 0.76 | 186.55 ± 26.25* | 201.33 ± 25.24*** | 121.67 ± 15.24*** |
| Compound D 0.76 | 270.41 ± 34.31* | 292.27 ± 29.44* | 78.43 ± 7.51*** |
| Compound E 0.76 | 206.18 ± 43.80*** | 160.27 ± 20.36*** | 89.62 ± 12.37*** |
| Compound F 0.76 | 221.58 ± 45.58*** | 157.39 ± 20.69*** | 105.02 ± 10.84*** |
| Compound G 0.76 | 225.60 ± 31.81*** | 152.22 ± 25.42*** | 99.52 ± 13.15*** |
| Compound H 0.76 | 230.15 ± 53.46*** | 167.23 ± 27.44*** | 105.52 ± 14.64*** |
| Compound I 0.76 | 214.27 ± 40.62*** | 159.82 ± 31.69*** | 115.02 ± 10.34*** |
| Compound J 0.76 | 268.28 ± 42.59*** | 197.45 ± 26.88*** | 95.67 ± 15.67*** |
| Nif 0.76 | 195.67 ± 32.63* | 166.68 ± 29.64*** | 183.15 ± 29.67*** |

*p < 0.05,
***p < 0.001, vs CMC-Na,

### EXPERIMENT EXAMPLE 4

### Protective Function to Cerebral Ischemia Model

1. Experiment Materials

NIH mice were supplied by the Animal Center of Medical Experiment, Guangdong Province, Certificate NO 98A032, 99A030; Tanakan was manufactured by Ipsen Company; malonic acid-(5-androsten-17-one-3β-ol)-diester (code-name compound C), succinic acid-(5-androsten-17-one-3β-ol)-diester (code-name compound D), glutaric acid-(5-androsten-17-one-3β-ol)-diester (code-name compound E), adipic acid-(5-androsten-17-one-3β-ol)-diester (code-name compound F), glycol-disulphonic acid-(5-androsten-17-one-3β-ol)-diester (code-name compound G), propanediol-disulphonic acid-(5-androsten-17-one-3β-ol)-diester (code-name compound H), butanediol-disulphonic acid-(5-androsten-17-one-3β-ol)-diester (code-name compound 1) and pentanediol-disulphonic acid-(5-androsten-17-one-3β-ol)-diester (code-name compound J) were synthesized by Medicine Chemistry Lab, Pharmacy Department of Zhongshan University. These were mixed with 0.5% CMC-Na to be different concentration when they were used.

2. Experiment Methods and Results

After weighed, NIH mice were anaesthetized with Penbotarbital 60 mg/kg, then performed operation. Fixed at supine position, the skin in the middle of the deck was dissected about 7 mm. Separated common carotid artery of two sides of neck, surrounded with the thread of No “0”. Common carotid artery of two sides was chipped with artery clip for 2 minutes to induce cerebral ischemia. In the end of ischemia treatment, artery clip was loosed and blood perfusion was restored, rest for five minutes, repeated twice as that. After blocking blood flow of carotid, sutured incision. Recorded trail bloodletting, bloodletting quantity was no more than 10% of total blood quantity. After operation, rats were administered with proper physiological saline through intraperitoneal injection in order to supple capacity of blood. After pseudo operation group was anaesthetized, carotid of two sides was separated but blood flow was not blocked, without being bloodletting too.

After being completely blockted carotid of two sides, mouse showed eclampsia first, lower body temperature and oliopnea and righting reflex disappeared in the end. In experiment appearance of body temperature lowering and righting reflex disappearing was as positive guide-line of ischemia, discarding those without typical guide line of ischemia. In the procedure of ischemia or after operation, some of animals would be dead. After cerebral ischemia-reperfusion, animals gradually restored righting reflex, hyperpnea, recovered and moved normally within 3-5 hours. After operation, animals were randomly divided into 11 groups as follows: pseudo operation group, group of compound C, D, E, F, G, H, I or J, Tanakan group and control group. According to the dose of Table 7, animals were administered with medicine by intragastric intubation once a day, and continue administered drug for 10 days. After 10 days, memory test was performed.

TABLE 7
Functions to memory step-through test of cerebral
ischemia-reperfusion injury (x ± s, n = 10)

| | | Latency of dark cabin | | |
|---|---|---|---|---|
| | | Study | Memory | Number of error |
| Group | Dose (mg · kg⁻¹) | (second) | (second) | (n) |
| Pseudo group | — | 32.7 ± 12.1** | 65.4 ± 15.2*** | 2.2 ± 1.0*** |
| Control group | — | 12.3 ± 5.2 | 33.1 ± 13.4 | 6.9 ± 1.4 |
| Compound C | 4.5 | 24.4 ± 17.3* | 58.1 ± 16.2*** | 2.5 ± 1.5*** |
| Compound D | 4.5 | 37.3 ± 15.1*** | 67.4 ± 19.7** | 1.2 ± 1.0*** |
| Compound E | 4.5 | 24.1 ± 16.0* | 45.8 ± 12.9* | 3.1 ± 2.7*** |
| Compound F | 4.5 | 28.7 ± 11.8*** | 46.5 ± 13.8* | 2.9 ± 1.1*** |
| Compound G | 4.5 | 29.5 ± 17.5* | 47.5 ± 15.7* | 3.4 ± 1.6*** |
| Compound H | 4.5 | 26.2 ± 15.2* | 56.0 ± 17.2* | 3.0 ± 1.5*** |
| Compound I | 4.5 | 25.7 ± 14.8* | 46.5 ± 13.8* | 3.1 ± 1.7*** |
| Compound J | 4.5 | 30.8 ± 15.0*** | 52.0 ± 17.2* | 3.2 ± 1.6*** |
| Tanakan | 31.2 | 23.1 ± 8.7** | 53.2 ± 18.6* | 3.3 ± 1.3*** |

*p < 0.05,
**p < 0.01,
***p < 0.001, vs CMC-Na,

The results in Table 7 showed that compared with control group, latency of entering dark cabin of pseudo operation group mouse was clearly prolonged and times attacked by electricity markedly decreased, indicating that cerebral ischemia-reperfusion had caused distinctly damage to study and memory function. Compared with control group, specimen could clearly prolong latency entering dark cabin, markedly decreased times of error and had protective function to injury of memory induced by cerebral ischemia.

### EXPERIMENT EXAMPLE 5

### Effect on the Immune Function

1. Experiment Materials

NIH mice, qualified-certificate NO: 97A018, were supplied by the Animal Center of Medical Experiment, Guangdong Province.

SD rats, animal qualified-certificate NO:97A107, were supplied by the Center of Experiment Animal, Guangdong Province; Domestic chickens were bought from food market in Zhongshan University; malonic diacid (5-androsten-17-one-3β-ol)diester (code-name compound C), succinic acid-(5-androsten-17-one-3β-ol)-diester (code-name compound D), glutaric acid-(5-androsten-17-one-3β-ol)-diester (code-name compound E), adipic acid-(5-androsten-17-one-3β-ol)-diester (code-name compound F), glycol-disulphonic acid-(5-androsten-17-one-3β-ol)-diester (code-name compound G), propanediol-disulphonic acid-(5-androsten-17-one-3β-ol)-diester (code-name compound H), butanediol-disulphonic acid-(5-androsten-17-one-3 β-ol)-diester (code-name compound 1) and pentanediol-disulphonic acid-(5-androsten-17-one-3β-ol)-diester (code-name compound J) were synthesized by medicine chemistry lab, Pharmacy Department of Zhongshan University. These were mixed with 0.5% CMC-Na to be different concentration when they were used.

2. Experiment Methods

Function to indexes of thymus and spleen of mouse.

Chose 90 healthy NIH mice, weighing (18˜22)g, equal amount of male and female. Every mouse was injected with cyclophos phamide (60 mg/kg, intraperitoneally). They were divided randomly into nine groups, ten mice in each group, equal amount of male and female, as follows: control group. compound C, D, E, F, G, H, I or J group. Administered specimen through intragastric intubation in terms of the dose in Table 8. Continuously administered orally specimen for 30 days, all mice fasted on the 30th day, but were supplied with water. On the 31th day, mice of every group were killed. Weighed their body, thymus and spleen, calculated index of thymus and spleen, compared the difference among each group.

3. Experiment Results

TABLE 8
Function to thymus and spleen of mice (x ± SD, n = 10)

| | | | Index of spleen |
|---|---|---|---|
| Group | | Index of thymus | (mg/10 g |
| Dose (mg/kg) | | (mg/10 g body weight) | body weight) |
| Control group | 1% CMC | 11.06 ± 3.31 | 42.42 ± 8.31** |
| Compound C | 10 | 18.98 ± 2.32*** | 57.55 ± 11.23*** |
| Compound D | 10 | 25.59 ± 3.70*** | 103.47 ± 13.40*** |
| Compound E | 10 | 20.22 ± 2.75*** | 68.20 ± 9.82*** |
| Compound F | 10 | 16.24 ± 3.31*** | 75.24 ± 15.48*** |
| Compound G | 10 | 18.39 ± 3.24*** | 80.22 ± 17.33*** |
| Compound H | 10 | 19.75 ± 3.06*** | 72.30 ± 11.17*** |
| Compound I | 10 | 16.44 ± 3.25*** | 69.60 ± 16.10*** |
| Compound J | 10 | 17.24 ± 3.34*** | 61.29 ± 15.34** |

**p < 0.01,
***p < 0.001, vs CMC-Na,

Known from the results of immune organ experiment of mice in Table 8, compared with control group, index of mouse thymus and spleen in group of compound C, D, E, F, G, H, I or J could remarkably enhance.

### EXPERIMENT EXAMPLE 6

### Pharmacological Effects to Osteoporosis

1. Experiment Animals

1.1 Animals

SD rats, equal male and female, weighing (196±47.3)_{g}, were supplied by the Animal Center of Medical Experiment, Guangdong Province.

1.2 Medicine and Agent

Malonic acid-(5-androsten-17-one-3β-ol)-diester (code-name compound C), succinic acid-(5-androsten-17-one-3β-ol)-diester (code-name compound D), glutaric acid-(5-androsten-17-one-3β-ol)-diester (code-name compound E), adipic acid-(5-androsten-17-one-3β-ol)diester (code-name compound F), glycol-disulphonic acid-(5-androsten-17-one-3β-ol)-diester (code-name compound G), propanediol-disulphonic acid-(5-androsten-17-one-3β-ol)-diester (code-name compound H), butanediol-disulphonic acid-(5-androsten-17-one-3β-ol)-diester (code-name compound I) and pentanediol-disulphonic acid-(5-androsten-17-one-3β-ol)-diester (code-name compound J) were synthesized by medicine chemistry lab, Pharmacy Department of Zhongshan University; Retinoic acid manufactured by Huabang Pharmacy Limited Company, Chongqin, Ratified Number (95), Medicine Authorized NO. X-140; Luogaiquan manufactured by Luo Pharmacy Limited Company, Shanghai, Ratified Number (96), Medicine Authorized NO. J-88-3(1), Certificate NO. B1063. The above medicine and agent were mixed with 0.5% CMC-Na to be different concentration before using.

1.3 Apparatus

XSP-BM bio-microscope was manufactured by Optics Apparatus Factory, Shanghai. Color Close-route Monitor of Panasonic was manufactured by Matushita Electrial Co., Ltd. Imagine analysis software of Chuangge was designed by Chuanggeke Group Company of CYTS. IRIS Advantage (HR, supplied by Measure and Test Center of Zhongshan University) was manufactured by Thermo Jarrell Ash Corporation, U.S.A.

2. Methods

2.1 Osteoporosis Model of Rats Induced by Retinoic Acid

Chose 88 rats, weighing (196±47.3)g, equal male and female, divided randomly into 11 groups, as follows: normal group, Retinoic acid model group (model group), positive control group (Rocaltrol group), specimen group of compound C, D, E, F, G, H, I or J. Except that normal group administered with 0.5% CMC-Na solution (10 mL/kg body weight, administered orally), each group rats were administered with Retinoic acid 70 mg/kg body weight in the morning and administered orally specimen in the afternoon. After continuously administering specimen for 30 days, killed rats and took right thighbone of rat.

2.2 Metrology Observation and Measure of Thighbone Tissue

Took same part tissue of right thighbone, routinely decalcified, enveloped with olefin, and dyed with HE. Observed with ordinary optics microscope, import all imagines into computer with A/D conversion of pickup camera, via dealing with image analysis software obtained index of guide line as follows: relative bone volume, indicating percentage that bone trabecula accounted for medullary cavity, 1.25×10×4 times; average width of bone trabecula, 1.25×10×10 times; amount of cell per area in bone trabecula, gathering same area image (640×480) of bone trabecula with image analysis software, calculating amount of bone cell in corresponding area, 1.25×10×40 times; Average length of bone cell lacuna, 1.25×10×40; Average width of bone cell lacuna, 1.25×10×40. The above five indexes of static state guide-line were all observed for ten eyeshots, calculating their average value.

TABLE 9
Effects on osteoporosis model of rats induced by Retinoic acid, n = 8, x ± s

| | | Amount of cell per | Average width of bone |
|---|---|---|---|
| Group | Relative bone volume/% | area in bone trabecula | trabecula/μm |
| Control group | 38.57 ± 3.25*** | 15.75 ± 4.21* | 68.61 ± 10.21** |
| Model group | 22.63 ± 3.15 | 12.71 ± 4.36 | 44.91 ± 18.72 |
| Rocaltrol group | 36.72 ± 7.82*** | 19.27 ± 3.89** | 67.16 ± 13.11** |
| Compound C | 38.94 ± 3.73*** | 19.50 ± 5.54* | 72.69 ± 10.38*** |
| Compound D | 41.55 ± 4.38*** | 20.70 ± 5.27*** | 70.76 ± 8.56*** |
| Compound E | 35.87 ± 3.12*** | 19.35 ± 3.13** | 68.66 ± 9.80*** |
| Compound F | 37.14 ± 3.46*** | 26.31 ± 3.16*** | 74.67 ± 3.56*** |
| Compound G | 34.37 ± 3.27*** | 25.84 ± 3.31*** | 69.67 ± 3.15** |
| Compound H | 38.31 ± 3.55*** | 35.69 ± 3.70*** | 60.84 ± 4.81* |
| Compound I | 29.84 ± 3.56*** | 31.84 ± 3.94*** | 61.37 ± 4.71* |
| Compound J | 37.55 ± 3.26*** | 29.84 ± 3.16*** | 67.84 ± 4.68* |

*p < 0.05,
**p < 0.01,
***p < 0.001, vs model group,

Known from results of osteoporosis of rats induced by Retinoic acid in Table 9, compared with model group, compound C, D, E, F, G, H, I and J could availably enhance relative bone volumes amount of cell per area in bone trabecula and average bone trabecula width, indicating that specimen contained function of curing osteoporosis.

### EXPERIMENT EXAMPLE 7

### Action to Renal Failure

1. Experiment Materials

1.1 Animal: NIH Mice, Weight (18˜22)g, Equal Amount of Male and Female, were Supplied by the Animal Center of Medical Experiment, Guangdong Province.

1.2 Agent: Endotoxin, Sigma Chemical Com.; Malonic acid-(5-androsten-17-one-3β-ol)-diester (code-name compound C), succinic acid-(5-androsten-17-one-3β-ol)-diester (code-name compound D), glutaric acid-(5-androsten-17-one-3β-ol)-diester (code-name compound E), adipic acid-(5-androsten-17-one-3β-ol)-diester (code-name compound F), glycol-disulphonic acid-(5-androsten-17-one-3β-ol)-diester (code-name compound G), propanediol-disulphonic acid-(5-androsten-17-one-3β-ol)-diester (code-name compound H), butanediol-disulphonic acid-(5-androsten-17-one-3β-ol)-diester (code-name compound I) and pentanediol-disulphonic acid-(5-androsten-17-one-3β-ol)-diester (code-name compound J) were synthesized by medicine chemistry lab, Pharmacy Department of Zhongshan University; The above medicine and agent were mixed with 0.5% CMC-Na to be different concentration before using.

2. Experiment Methods

Acute renal failure model of animal induced by endotoxin

Chose 100 mice, divided randomly into 10 groups as follows: normal group, pathology model group, specimen group of compound C, D, E, F, G, H, I or J. After respectively administered with specimen or same volume solvent (0.5% CMC-Na) by intraperitoneal injection, each group was immediately injected with endotoxin 0.06 g/kg (normal group was injected with same volume saline) through trail vein. After five hours, blood of the mice was collected by removing eyes, assayed Blood Urea Nitrogen (BUN) and Creatinine (Cr) concentration of serum. One kidney of each rat was taken out and made 100 mL/L homogenate, assayed activity of Glutathione Peroxidase (GSH-Px) enzyme.

3. Experiment Results

TABLE 10
Function of specimen to acute renal failure (x ± SD, n = 10)

| Group | Dose(mg/kg) | Cr/10 mg · L⁻¹ | BUN/10 mg · L⁻¹ | GSH-Px/nU · L⁻¹ |
|---|---|---|---|---|
| Normal group | 0.5% CMC | 12.28 ± 2.53*** | 182.5 ± 26.9*** | 45.5 ± 18.7*** |
| Pathology group | 0.5% CMC | 59.23 ± 13.15 | 485.8 ± 34.7 | 19.3 ± 8.4 |
| Compound C | 10 | 37.99 ± 8.74*** | 387.0 ± 40.2*** | 29.8 ± 11.6* |
| Compound D | 10 | 29.31 ± 7.94*** | 295.5 ± 36.0*** | 38.4 ± 15.0** |
| Compound E | 10 | 32.86 ± 8.07*** | 315.9 ± 40.8*** | 32.0 ± 14.4* |
| Compound F | 10 | 38.31 ± 9.69*** | 377.1 ± 38.1*** | 35.2 ± 12.8* |
| Compound G | 10 | 40.42 ± 10.94*** | 412.2 ± 40.5*** | 31.8 ± 12.3* |
| Compound H | 10 | 35.78 ± 7.26*** | 366.0 ± 30.2*** | 35.6 ± 10.7* |
| Compound I | 10 | 34.41 ± 9.65*** | 344.4 ± 40.6*** | 37.6 ± 15.1*** |
| Compound J | 10 | 37.60 ± 8.44*** | 337.6 ± 32.8*** | 32.2 ± 13.1** |

*p < 0.05,
**p < 0.01,
***p < 0.001, vs pathology group,

The experiment results of acute renal failure showed in Table 10, compared with pathology group, compounds C, D, E, F, G, H, I or J could availably inhibit enhancing of BUN and Cr of mice induced by endotoxin, increased activity of GSH-Px enzyme and had protective action to acute renal failure of animals induced by endotoxin.

Above experiment examples of this invention indicated that compounds I had remarkable therapeutic effects on arrhythmia, myocardial ischemia, cerebral ischemia, low immunity function, osteoporosis, renal failure and so on and could be used to manufacture drugs for these diseases.

1. The application of diacid-(5-androsten-17-one-3β-ol)-diester (formula I) was used to manufacture drug to treat arrhythmia, myocardial ischemia, cerebral ischemia, osteoporosis, renal failure and improve immunity in pharmacy fields. [chemical structure]
